(19) United States
(12) Patent Application Publication
(10) Pub. No.: US 2025/0059144 A1
(43) Pub. Date: Feb. 20, 2025

(54) Tellurium-containing compounds and the use of treating bacterial infections thereof

(71) Applicant: Kaohsiung Medical University, Kaohsiung City (TW)

(72) Inventors: Sung-Pin Tseng, Kaohsiung City (TW); Tsung-Ying Yang, Pingtung County (TW); Po-Liang Lu, Kaohsiung City (TW)

(73) Assignee: Kaohsiung Medical University, Kaohsiung City, TW (TW)

(21) Appl. No.: 18/715,132

(22) PCT Filed: Dec. 2, 2022
(86) PCT No.: PCT/CN2022/136153

§ 371 Date: May 31, 2024

Related U.S. Application Data
(60) Provisional application No. 63/286,100, filed on Dec. 6, 2021.

(51) Int. Cl. C07D 293/10; A61K 31/357; A61K 31/4184; A61K 31/437; A61K 31/554; A61P 31/04; C07D 329/00; C07D 517/14

(52) U.S. Cl. CPC C07D 293/10; A61K 31/357; A61K 31/4184; A61K 31/437; A61K 31/554; A61P 31/04; C07D 329/00; C07D 517/14

(57) ABSTRACT

A compound and the method for treating bacterial infections thereof are provided, wherein the compound is derived from AS101 and having a general formula (I) or a general formula (II), and wherein the method includes administering an effective amount of compound having the general formula (I) or a compound having the general formula (I) and its pharmaceutically acceptable salt thereof.

### CROSS-REFERENCE TO RELATED APPLICATION

This application is a national stage application, filed under 35 U.S.C. § 371, of International Patent Application No. PCT/CN2022/136153 filed on Dec. 2, 2022, and claims the benefit of priority from U.S. Provisional application No. 63/286,100 filed on Dec. 6, 2021, the contents of each of which is incorporated by reference herein in its entirety.

### FIELD OF THE INVENTION

The present invention relates to a tellurium-containing compounds and the use of treating bacterial infections thereof.

### BACKGROUND OF THE INVENTION

The discovery, development and research of the potent and safe antibiotics have brought about major improvements to healthcare. However, the efficacy of the antibiotic treatment is currently limited due to the limited available agents and their bacterial resistance. Hence, the demand for the development of novel antibacterial agents is growing since the pathogens are difficult to treat currently with antibiotic resistance. Such pathogens may include Enterobacteriaceae, Acinetobacter baumannii, Pseudomonas aeruginosa.

Klebsiella pneumoniae, one of pathogen classified as Enterobacteriaceae, can easily cause nosocomial infections in the intensive care unit of hospital and the communities. It can generally be found in the respiratory tract or gastrointestinal tract of healthy people. Individuals with weakened immunity may cause severe infections, including pneumonia, bacteremia, meningitis, liver abscess, endophthalmitis, inflammation disorder of urinary system, or wound infections. The mortality rate is extremely high if there is an improper treatment. Patients are mostly treated with β-lactam antibiotics, including cephalosporin and carbapenem.

Another representative case is Escherichia coli, a gram-negative facultative anaerobic bacterium, which also belongs to the Enterobacteriaceae family. Although most strains are harmless and can coexist with the human body, some of the infectious pathogenic Escherichia coli could still carry specific virulence factors in clinical practice, which lead to gastroenteritis, hemorrhagic colitis and other ex-intestinal infections (including urinary tract infections, bacteremia and other symptoms). β-lactams (such as cephalosporins and carbapenems) are the preference antibiotic for the above infections as the same treatment of the Klebsiella pneumoniae infection. Most of the symptoms of extra-intestinal infections can be alleviated by β-lactams treatment.

However, according to the previous reports, β-lactamase plays the essential role of the mechanism of drug resistance. The enzyme can hydrolyze β-lactams of antibiotic drugs. In addition, the genes of the β-lactamase mostly locate in plasmids or transposon, which causes the rapid spread of drug resistant genes between bacteria. The extended-spectrum β-lactamases (ESBL) can further hydrolyze a variety of β-lactams antibiotics, including all penicillins series, the third-generation cephalosporins (cefotaxime, ceftriaxone, ceftazidime) and the previous series.

Acinetobacter baumannii, an aerobic, non-lactose-fermented gram-negative pathogen, can exist in the vigorous environment for a long time with no flagella and mobility. Acinetobacter baumannii is one of the common pathogens of nosocomial infections, particular in the intensive care unit. It usually causes bacteremia, pneumonia, meningitis, peritonitis, endocarditis, and urinary tract and skin infections. The treatment of Acinetobacter baumannii infection is relatively difficult to cure because the bacterium has a variety of endogenous drug resistance. Furthermore, it is easy for Acinetobacter baumannii to obtain drug resistance through plasma, transposon, and integron conjugation. In fact, Acinetobacter baumannii has resistance to the third and fourth generation cephalosporins (such as ceftazidime and cefepime, etc.). Only 20% to 40% of Acinetobacter baumannii are susceptible to cephalosporin in the world. Therefore, the broad spectrum cephalosporin is not suitable for the clinical treatment of Acinetobacter baumannii infection, and could only be used after the examinations of the drug sensitivity test to confirm its susceptibility. In view of the cephalosporin resistance of Acinetobacter baumannii, carbapenem alone can be used as a preferred option for the treatment of Acinetobacter baumannii infection. However, due to the rise of carbapenem resistance, the success rate of clinical therapy with carbapenem alone has decreased. As a result, combination antibiotic therapy with carbapenem are given to temporarily alleviate the problem, such as such as colistin, tigecycline, etc., which implied the necessity of developing new drugs for carbapenem resistance.

Pseudomonas aeruginosa is a facultative anaerobic gram-negative bacillus with flagella and motility. It can produce the smell of grapes in the culture medium an secrete a variety of pigments, including pyocyanin (cyan), pyoverdin (fluorescent yellow) and pyorubin (brownish red). Additionally, it can grow in the extreme environments, such as diesel, 42° C. environment. Pseudomonas aeruginosa is one of the common pathogens of nosocomial infections, which cause lungs, urinary tract, burn wounds, blood infections, and even corneal infections when the contact lenses are not cleaned. For those who are suspected Pseudomonas aeruginosa infections, third and fourth generation of the cephalosporins (such as ceftazidime and cefepime, etc.) or carbapenems (such as imipenem) with gentamicin, amikacin or ciprofloxacin can be used as the initial treatment in the clinical practice firstly. After the results of the susceptibility test in the microbiological testing is acquired, the therapy strategy might be accordingly changed.

Severe infections of broad spectrum β-lactamase-producing Enterobacteriaceae can be treated with carbapenem. Recently, with the rise of the use of carbapenems, the use of late-line antibiotic increase annually, which cause serious plight in the clinical practice that the infection will be difficult to treat. The mechanism of carbapenem resistance includes overexpression of the AmpC gene, alteration in ESBL combined with membrane protein (porin), and the acquisition of carbapenemase that can decompose carbapenem, such as Klebsiella pneumoniae carbapenemase (KPC) and New Delhi metallo-β-lactamase 1 (NDM-1). Since most of the carbapenemase genes mentioned above, such as KPC and NDM-1, are located on the plastids, the resistance genes are easily spread among bacteria through the plasmid conjugation. Those carbapenem-resistant Enterobacteriaceae strains (carbapenem-resistant Enterobacteriaceae); CRE) may lead to limited treatment options.

The carbapenem resistance of Acinetobacter baumannii is mainly attributed to reducing of membrane permeability (loss of the channel protein porin) or enhancement of expression of the efflux pump. In addition, carbapenemase can also cause the carbapenem resistance of Acinetobacter baumannii infection. Apart from case of Enterobacteriaceae that the carbapenemase mainly belongs to class A (KPC) or class B (NDM), the carbapenemase carried by Acinetobacter baumannii are mainly OXA-23 and its variants which are belonging to class D. carbapenemase inhibitors are one of the main targets for the development of emerging antibiotics recently (i.e. β-lactamase inhibitors, such as avibactam or relebactam, etc.). Although the common carbapenemase OXA-48 in Enterobacteriaceae can be inhibited by avibactam and relebactam, the common carbapenemase of Acinetobacter baumannii cannot be inhibited by avibactam and relebactam. At present, the proportion of carbapenem resistance of Acinetobacter baumannii is rapidly increasing globally, especially in Europe. 80% of hospital care-related Acinetobacter baumannii infections in Europe are carbapenem-resistant strains. Thus, the development of new drugs for carbapenem-resistant Acinetobacter baumannii is even more important.

The carbapenem resistance mechanism of Pseudomonas aeruginosa is similar to Acinetobacter baumannii that mainly due to the loss of channel protein (porin) or the overexpression of efflux pump that combined with cephalosporinase AmpC overexpression. According to the previous investigations, about 20% of the strains contained carbapenemase in Europe in 2011 and only 4.3% in Canada in 2017. Most of them belonged to the class B (such as VIM, IMP, etc.). However, due to the transferability of carbapenemase (located in plasmids or transposons), the proportion of Pseudomonas aeruginosa with carbapenemase has gradually increased and reached to 70-88%. In addition, cases of Pseudomonas aeruginosa carrying GES carbapenemase (class A) have been increasing annually, which leads to the popularity of carbapenemase in Pseudomonas aeruginosa, thereby increasing the popularity of carbapenem resistance.

In 2017, the WHO listed carbapenem-resistant Enterobacteriaceae, Pseudomonas aeruginosa, and Acinetobacter baumannii as drug-resistant bacteria that urgently need the development of novel antibiotics. Hence, there exists an urgent demand for developing novel and effective antimicrobial agents, particularly those bacteria that exhibit resistance to the aforementioned antibiotics.

### BRIEF DESCRIPTION OF THE DRAWINGS

FIG. 1 are the formulae of the compounds (a), (b), (c), (d), (e), (f), (g), and (h).

FIG. 2 illustrates an example of the 50% lethal dose (LD₅₀) of compound (c) to mice.

FIG. 3 illustrates the survival curve of compound (c) in the treatment of carbapenem-resistant Klebsiella pneumoniae sepsis infection model.

FIG. 4 illustrates the organ bacteria counts of the compound (c) treatment of carbapenem-resistant Klebsiella pneumoniae sepsis infection model.

FIG. 5 illustrates the survival curve of compound (c) in the treatment of carbapenem-resistant Acinetobacter baumannii sepsis infection model.

### DETAILED DESCRIPTION OF THE INVENTION

As used herein, “a”, “an”, “the”, “at least one”, and “one or more” are used interchangeably.

The present invention is a compound having the following general formula (I):

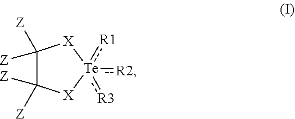

wherein R1, R2 and R3 are all halogens, or two of R1, R2 and R3 are halogens, and another one is none, wherein “ [symbol] ” indicates bonding or none; wherein the X—R4 is N or O, and R4 is Hydrogen or alkyl; wherein the Z is Hydrogen (H) or Deuterium (D), or the Z is together fused with a phenyl to form a benzene; and wherein the Z is not H while the X is O.

The present invention is related to a pharmaceutical composition comprising an effective amount of compound having the following general formula (I) or a compound having the following general formula (I) and its pharmaceutically acceptable salt thereof:

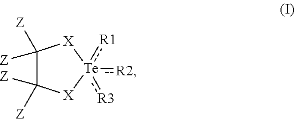

wherein R1, R2 and R3 are all halogens, or two of R1, R2 and R3 are halogens, and another one is none, wherein “ [symbol] ” indicates bonding or none; wherein the X is N or O; wherein the Z is H or D, or the Z is together fused with a phenyl to form a benzene; and wherein the Z is not H while the X is O.

The present invention is also related to a use of a pharmaceutical composition in preparation of a medicament for treating bacterial infections, wherein the pharmaceutical composition comprising an effective amount of compound having the following general formula (I) or a compound having the following general formula (I) and its pharmaceutically acceptable salt thereof:

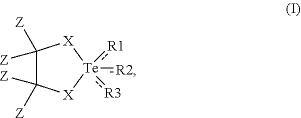

wherein R1, R2 and R3 are all halogens, or two of R1, R2 and R3 are halogens, and another one is none, wherein “ [symbol] ” indicates bonding or none; wherein the X is N—R4 or O, and R4 is Hydrogen or alkyl; wherein the Z is H or D, or the Z is together fused with a phenyl to form a benzene; and wherein the Z is not H while the X is O.

The present invention is also related to a method for treating a subject suffering from bacterial infections comprising administering a pharmaceutical composition to the subject, wherein the pharmaceutical composition comprising an effective amount of compound having the following general formula (I) or a compound having the following general formula (I) and its pharmaceutically acceptable salt thereof.

The present invention is further related to a use of a pharmaceutical composition in preparation of a medicament for treating at least one disease caused by the bacterial infections, wherein the pharmaceutical composition comprising an effective amount of compound having the following general formula (I) or a compound having the following general formula (I) and its pharmaceutically acceptable salt thereof:

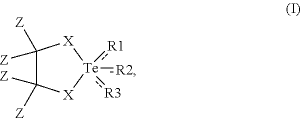

wherein R1, R2 and R3 are all halogens, or two of R1, R2 and R3 are halogens, and another one is none, wherein “ [symbol] ” indicates bonding or none; wherein the X is N—R4 or O, and R4 is Hydrogen or alkyl; wherein the Z is H or D, or the Z is together fused with a phenyl to form a benzene; and wherein the Z is not H while the X is O.

The present invention is also related to a method for treating a subject suffering from at least one disease caused by bacterial infections comprising administering a pharmaceutical composition to the subject, wherein the pharmaceutical composition comprising an effective amount of compound having the following general formula (I) or a compound having the following general formula (I) and its pharmaceutically acceptable salt thereof.

In an embodiment of the present invention, wherein the compound is in a salt form and has the following general formula (I-1):

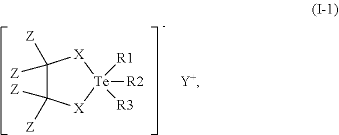

wherein R1, R2 and R3 are halogens; wherein the X is N—R4 or O, and R4 is Hydrogen or alkyl; wherein Y is ammonium, phosphonium, potassium, sodium or lithium; wherein the Z is Hydrogen or Deuterium; and wherein the Z is not H while the X is O.

In a preferred embodiment of the present invention, wherein the compound is compound (a)

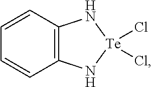

compound (c)

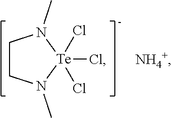

or compound (d)

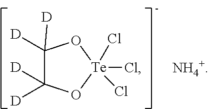

In the most preferred embodiment of the present invention, wherein the compound is compound I

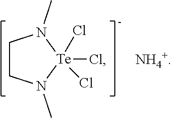

The present invention is a compound having the following general formula (II):

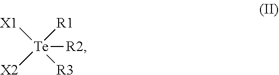

wherein R1, R2 and R3 are all halogens; and wherein the X1 and X2 are together bipyridine, dipyridinyl disulfide or phenanthroline, wherein the bipyridine is unsubstituted or substituted by one or more alkyl substituents.

The present invention is related to a pharmaceutical composition comprising an effective amount of compound having the following general formula (II) or a compound having the following general formula (II) and its pharmaceutically acceptable salt thereof:

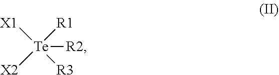

wherein R1, R2 and R3 are all halogens; and wherein the X1 and X2 are together bipyridine, dipyridinyl disulfide or phenanthroline, wherein the bipyridine is unsubstituted or substituted by one or more alkyl substituents.

The present invention is also related to a use of a pharmaceutical composition in preparation of a medicament for treating bacterial infections, wherein the pharmaceutical composition comprising an effective amount of compound having the following general formula (II) or a compound having the following general formula (II) and its pharmaceutically acceptable salt thereof:

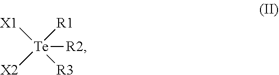

wherein R1, R2 and R3 are all halogens; and wherein the X1 and X2 are together bipyridine, dipyridinyl disulfide or phenanthroline, wherein the bipyridine is unsubstituted or substituted by one or more alkyl substituents.

The present invention is also related to a method for treating a subject suffering from bacterial infections comprising administering a pharmaceutical composition to the subject, wherein the pharmaceutical composition comprising an effective amount of compound having the following general formula (II) or a compound having the following general formula (II) and its pharmaceutically acceptable salt thereof.

The present invention is further related to a use of a pharmaceutical composition in preparation of a medicament for at least one disease caused by the bacterial infections, wherein the pharmaceutical composition comprising an effective amount of compound having the following general formula (II) or a compound having the following general formula (II) and its pharmaceutically acceptable salt thereof:

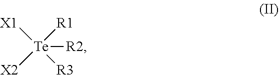

wherein R1, R2 and R3 are all halogens; and wherein the X1 and X2 are together bipyridine, dipyridinyl disulfide or phenanthroline, wherein the bipyridine is unsubstituted or substituted by one or more alkyl substituents.

The present invention is also related to a method for treating a subject suffering from at least one disease caused by bacterial infections comprising administering a pharmaceutical composition to the subject, wherein the pharmaceutical composition comprising an effective amount of compound having the following general formula (II) or a compound having the following general formula (II) and its pharmaceutically acceptable salt thereof.

In an embodiment of the present invention, wherein the compound is in a salt form and has the following general formula (II-1):

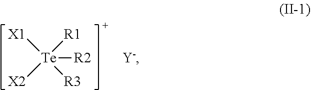

wherein R1, R2 and R3 are all halogens; wherein Y is halogen; and wherein the X1 and X2 are together bipyridine, dipyridinyl disulfide or phenanthroline, wherein the bipyridine is unsubstituted or substituted by one or more alkyl substituents.

In a preferred embodiment of the present invention, wherein the compound is compound (b)

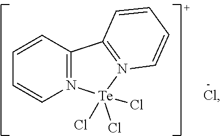

compound (e)

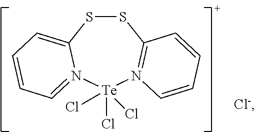

compound (f)

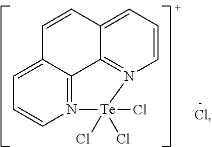

compound (g)

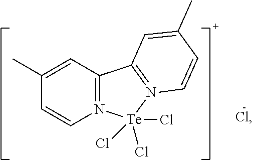

or compound (h)

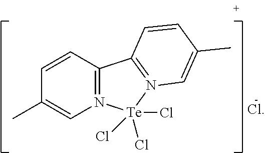

In one embodiment of the present invention, wherein the bacterial infections are caused by Gram-negative bacteria.

Preferably, wherein the bacterial infections are caused by Klebsiella pneumoniae, Escherichia coli, Pseudomonas aeruginosa, Acinetobacter baumannii, Elizabethkingia meningoseptica, Neisseria gonorrhoeae and/or Enterobacter spp. complex.

In one embodiment of the present invention, wherein the disease is selected from one or more of respiratory tract infection, urinary tract infection, central nervous system infection, ear infection, pleuropneumonia and bronchial infection, intra-abdominal infection, cardiovascular infection, skin or soft tissue infections, bone and joint infections, genital infection, eye infection, pharyngeal infection, and oral infection.

In another embodiment of the present invention, wherein the disease is selected from one or more of upper respiratory tract infection, lower respiratory tract infection, tracheitis, bronchitis, pneumonia, pulmonary tuberculosis, pharyngitis, complicated urinary tract infection, non-complicated urinary tract infections, cystitis, pyelonephritis, encephalitis, meningitis, brain abscess, otitis externa, otitis media, blood infection, endocarditis, myocarditis, pericarditis, arthritis, osteomyelitis, genital ulceration, vaginitis, cervicitis, conjunctivitis, keratitis, endophthalmitis, and gingivitis.

Preferably, wherein the blood infection is sepsis or bacteremia.

### Embodiments

One skilled in the art readily appreciates that the present invention is well adapted to carry out the objects and obtain the ends and advantages mentioned, as well as those inherent therein. The compositions, processes and methods for producing them, and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

The following examples are not intended to be limiting and are only used to present various aspects of the present invention. To make the above-described and other purposes, features, and advantages of the present invention more obvious and easier to understand, preferred embodiments are provided below, and are described in detail as follows.

### EXAMPLES

The present invention provides a library of tellurium-containing structure derivatives. The synthesis process are as follows.

Example 1: Synthesis of the Tellurium-Containing Compounds

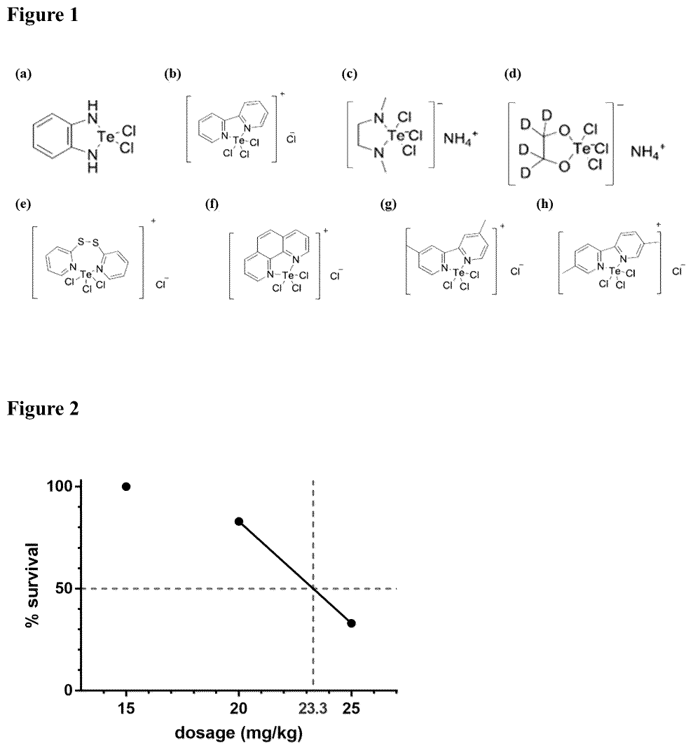

The present invention used the following synthesis method to synthesis AS101 analog compounds, all the structure of compounds are shown in FIG. 1.

### Compound (a)

TeCl₄ (0.4 mmol) and o-Phenylenediamine (2.0 mmol) were dissolved in toluene (3 ml) respectively. After TeCl₄ and o-Phenylenediamine were dissolved completely, the TeCl₄ solution was added into the o-Phenylenediamine solution, solid products were formed and were collected. Then, bromobenzene was added with continuous stirring to remove the excess hydrochloride as by-product, compound (a) was obtained.

### Compound (b)

TeCl₄ (0.4 mmol) and 2,2′-Bipyridine (0.4 mmol) were dissolved in THF (3 mL) respectively. After TeCl₄ and 2,2′-Bipyridine were dissolved completely, the TeCl₄ solution was added into the Bipyridine solution, and then solid products were formed. Continuously stirring the mixed solution for 1-2 h, the mixed solutions were placed at −30° C. for an hour. The solid products were collected and were dehydrated, compound (b) was therefore obtained.

### Compound (c)

TeCl₄ (0.4 mmol) and N,N′-Dimethylethylenediamine (2.0 mmol) were dissolved in ACN (3 ml) respectively. After TeCl₄ and N,N′-Dimethylethylenediamine were dissolved completely, the TeCl₄ solution was added into the N,N′-Dimethylethylenediamine solution. The mixed solution was stirred for an hour at room temperature, and solid products were collected and were dehydrated as compound (c).

### Compound (d)

TeCl₄ (0.4 mmol) and Ethylene-d14 glycol (2.0 mmol) were placed in the round-bottomed flask, dry ACN (3 ml) was added and nitrogen was filled to heat-reflux for 16 hours. Cool the mixed solution to room temperature, the formed solid products were collected. Washed the solid products by ACN and dry the washed products to obtain compound (d).

### Compound (e)

TeCl₄ (0.4 mmol) and 1,2-di(pridin-2-yl)disulfane (0.4 mmol) were dissolved in THF (3 mL) respectively. After TeCl₄ and 1,2-di(pridin-2-yl)disulfane were dissolved completely, the TeCl₄ solution was added into the Bipyridine solution, and then solid products were formed. Continuously stirring the mixed solution for 1-2 h, the solutions were placed at −30° C. for an hour. The solid products were collected and were dehydrated as compound (e).

### Compound (f)

TeCl₄ (0.4 mmol) and 1,10-phenanthroline (0.4 mmol) were dissolved in THF (3 mL) respectively. After TeCl₄ and 1,10-phenanthroline were dissolved completely, the TeCl₄ solution was added into the Bipyridine solution, and then solid products were formed. Continuously stirring the mixed solution for 1-2 h, the mixed solutions were placed at −30°° C. for an hour. The solid products were collected and were dehydrated as compound (f).

### Compound (g)

TeCl₄ (0.4 mmol) and 4-4′-dimethyl-2,2′-bipyridine (0.4 mmol) were dissolved in THF (3 mL) respectively. After TeCl₄ and 4-4′-dimethyl-2,2′-bipyridine were dissolved completely, the TeCl₄ solution was added into the Bipyridine solution and then solid products were formed. Continuously stirring the mixed solution for 1-2 h, the mixed solutions were placed at −30° C. for an hour. The solids were collected and dehydrated as the compound (g).

### Compound (h)

TeCl₄ (0.4 mmol) and 5,5′-dimethyl-2,2′-bipyridine (0.4 mmol) were dissolved in THF (3 mL) respectively. After TeCl₄ and 5,5′-dimethyl-2,2′-bipyridine were dissolved completely, the TeCl₄ solution was added into the Bipyridine solution and then solid products were formed. Continuously stirring the mixed solution for 1-2 h, the mixed solutions were placed at −30° C. for an hour. The solids were collected and dehydrated as the compound (h).

### Example 2: Selection of Potential Candidate Compounds

In order to select the compounds for further investigations, Cation-adjusted Muller-Hinton broth (CAMHB) was used to evaluate the Minimum Inhibitory Concentration (MIC) values of the compounds (a), (b), (c), (d), (e), (f), (g) and (h), As shown in Table 1, compounds (a) and (c) exhibits better anti-infection bioactivity, especially the compound (c), which shows significant MIC decrease in infections of Klebsiella pneumoniae (ATCC BAA-1705), Klebsiella pneumoniae (strain CRE-723), Escherichia coli (CRE-415), Pseudomonas aeruginosa (strain PA13), Acinetobacter baumannii (strain AB03), Neisseria gonorrhoeae (ATCC 19424), Enterobacter complex (clinical isolate Nov. 8, 1950) and Elizabethkingia meningoseptica (clinical isolate E36), reveals much better anti-bacterial activities. Compound (c) was therefore used for further evaluation of anti-bacterial activities to carbapenem resistant bacteria involving Escherichia coli, Klebsiella pneumoniae, Pseudomonas aeruginosa, and Acinetobacter baumannii in the following experiments.

TABLE 1
The MIC comparison of analog compounds (a), (b), (c), (d), (e),
(f), (g), and (h) in five carbapenem resistant bacteria.

| | MIC (μg/mL) | | | | | | | |
|---|---|---|---|---|---|---|---|---|
| | K. | K. | E. | P. | A. | | | |
| | pneumoniae | pneumoniae | coli | aeruginosa | baumannii | Neisseria | Enterobacter | Elizabethkingia |
| | ATCC | strain | CRE- | strain | strain | gonorrhoeae | complex | meningoseptica |
| Analogs | BAA-1705 | CRE-723 | 415 | PA13 | AB03 | ATCC 19424 | 11-08-50 | E36 |
| (a) | 4 | 2 | 1 | 2 | 1 | ND | ND | ND |
| (b) | 16 | 16 | 4 | 4 | 2 | ND | ND | ND |
| (c) | 2 | 2 | <0.5 | 1 | <0.5 | 0.125 | 0.5 | 0.5 |
| (d) | 8 | 8 | 1 | 1 | 1 | ND | ND | ND |
| (e) | 16 | 32 | 4 | 16 | 4 | ND | ND | ND |
| (f) | 8 | 4 | 8 | 2 | 1 | ND | ND | ND |
| (g) | 16 | 16 | 16 | 4 | 4 | ND | ND | ND |
| (h) | 8 | 16 | 4 | 8 | 4 | ND | ND | ND |

34 carbapenem-resistant Klebsiella pneumoniae, 34 carbapenem-resistant Escherichia coli, 29 carbapenem-resistant Pseudomonas aeruginosa isolated from infected blood, and 28 carbapenem-resistant Acinetobacter baumannii isolated from infected blood were selected to evaluate the anti-bacterial activities of compounds (c). As shown in the Table 2, the MIC range, the MIC₅₀ and the MIC₉₀ of compound (c) to the Acinetobacter baumannii is <0.5˜4, 1 and 2 μg/mL, respectively, which is significantly lower than those of AS101 to the Acinetobacter baumannii (p<0.0001). The MIC range, the MIC₅₀ and the MIC₉₀ of compound (c) to the Escherichia coli is <0.5˜>64, 2 and 4 μg/mL, respectively, which is significantly lower than those of AS101 to the Escherichia coli (p<0.0001). The MIC range, the MIC₅₀ and the MIC₉₀ of compound (c) to the Klebsiella pneumoniae is <0.5>8, 2 and 8 μg/mL, respectively, which is significantly lower than those of AS101 to the Klebsiella pneumoniae (p<0.0001). The MIC range, the MIC₅₀ and the MIC₉₀ of compound (c) to the Pseudomonas aeruginosa is <0.5˜>64, 16 and >64 μg/mL, respectively, which is similar to AS101 to the Pseudomonas aeruginosa.

TABLE 2
The MICs of compounds (c) in four carbapenem resistant bacteria.

| Bacterial spp. | Compound | MIC₅₀ | MIC₉₀ | MIC range |
|---|---|---|---|---|
| A. baumannii (n = 28) | AS101 | 2 | 4 | <0.5~8 |
| | (c) | 1 | 2 | <0.5~4 |
| E. coli (n = 34) | AS101 | 4 | 8 | 0.5~32 |
| | (c) | 2 | 4 | <0.5~8 |
| K. pneumoniae (n = 34) | AS101 | 2 | 16 | <0.5~16 |
| | (c) | 2 | 8 | <0.5~8 |
| P. aeruginosa (n = 29) | AS101 | 64 | >64 | <0.5~>64 |
| | (c) | 16 | >64 | <0.5~>64 |

### Example 3: Acute Toxicity Test

In terms of acute toxicology, the dose-response relationship is represented in S-shaped curve. It is not easy to obtain the LD₅₀ value directly through the experiment. Therefore, the animal survival rate of 20˜80% must be used as the benchmark, and the LD₅₀ value is obtained by calculating the dose and survival rate of the area. The results of acute toxicity test were shown in FIG. 2. Six male mice of each group were injected with 15, 20, and 25 mg/kg of compound (c) by intraperitoneal injection. After a week of behavior and physiological observations, the survival rate was recorded and applied for linear regression.

The survival rate in 15 mg/kg administered groups were 100% (6/6), the survival rate in 20 mg/kg administered groups were 83.3% (5/6), and the survival rate in 25 mg/kg administered groups were 33.3% (2/6). Hence, the survival data of 20 mg/kg administered groups and 25 mg/kg administered groups were applied for linear regression and then obtained the calculated formula of Y (survival rate)=2.83−0.1× (dose). The LD₅₀ value is 23.3 mg/kg.

The end of the experiment was the 7^{th} day after administration, and all the surviving mice had good activity and the weight was significantly recovered significantly. In the experiment, 2 mice in the 20 and 25 mg/kg groups were randomly selected for euthanasia, the tissues involving heart, liver, spleen, lung, kidney, stomach, small intestine, large intestine, pancreatic were sliced and stained and were used for histopathological lesion evaluation and severity score by a professional pathology veterinarian (Disease Animal Special Certificate No. 0040). The results were shown in Table 3.

TABLE 3
Histopathological lesion evaluation and severity score of the surviving mice.

| | | Animal code and dosage ^{b} | | | |
|---|---|---|---|---|---|
| | | 20-1 | 20-2 | 25-1 | 25-2 |
| Organ | Histopathological changes | 20 mg/kg | | 25 mg/kg | |
| Heart | Necrosis/ degeneration | 0 | 0 | 0 | 0 |
| | Mononuclear cells infiltration | 0 | 0 | 1 | 0 |
| Liver | Mononuclear cells infiltration | 1 | 0 | 1 | 1 |
| | Necrosis/ degeneration | 0 | 0 | 0 | 1 |
| | Glycogen deposition | 3 | 4 | 1 | 3 |
| | Increased mitosis | 0 | 0 | 0 | 0 |
| | Extramedullary hematopoiesis, EMH | 1 | 2 | 0 | 1 |
| Spleen | Extramedullary hematopoiesis, EMH | 2 | 2 | 2 | 1 |
| | Apoptosis | 0 | 0 | 0 | 1 |
| | Necrosis | 0 | 0 | 0 | 0 |
| Lung | Mononuclear cells infiltration | 0 | 0 | 0 | 0 |
| | Necrosis/ degeneration | 0 | 0 | 0 | 0 |
| Kidney | Nephropathy, chronic progressive (CPN) ^{a} | 3 | 4 | 3 | 0 |
| Stomach | Mononuclear cells infiltration | 0 | 0 | 0 | 0 |
| | Erosion/ ulcer | 0 | 0 | 0 | 0 |
| Small | Mononuclear cells infiltration | 0 | 0 | 0 | 0 |
| intestine | Erosion/ ulcer | 0 | 0 | 0 | 0 |
| Large | Mononuclear cells infiltration | 0 | 0 | 0 | 0 |
| intestine | Erosion/ ulcer | 0 | 0 | 0 | 0 |
| Pancreas | Mononuclear cells infiltration | 0 | 0 | 0 | 0 |
| | Necrosis | 0 | 0 | 0 | 0 |

^{a} CPN, including lesions of tubular basophilia, basement membrane thickening, mononuclear inflammatory cells infiltration glomerulosclerosis.
^{b} 20-1, 20-1 indicate 2 mice in the 20 mg/kg administered group; 25-1, 25-2 indicate 2 mice in the 25 mg/kg administered groups.

As shown in Table 3, no significant myocardial degeneration or necrosis was observed in the heart. Small amounts of monocyte infiltration were observed in the mouse No. 25-1, and none of the lesion was observed in the others. Extremely light to slight monocyte infiltration in the heart was a lesion that were occasionally observed by a normal mouse might be irrelevant to experimental disposal, and a small number of monocyte infiltration cells could be observed in the portal vein area of the liver of most mice. Mild to moderate hepatocyte glycogen deposition were found in all mice, and significant swollen occurred in their liver cells, this was a common background lesions in normal mice in the case of sacrifice without sufficient fasting. It was worth noting that slight necrosis of the liver was only seen in No. 25-2. Increased mitosis in liver cell is one of the common regeneration or hyperplasia reactions for liver tissue damage. Compared with normal liver tissue, damaged liver tissue may have more mitosis cells, but it is also occasionally seen in normal mice. The mice of Nos. 20-1 and 25-2 had extremely slight to mild hepatocytes increased mitosis. It might be irrelevant to experimental disposal since the degree of increased mitosis was extremely mild to mild and was not related to the dose. Extramedullary hematopoiesis (EMH) are common lesions of mice liver tissue, it can be considered as background lesion if the degree is extremely mild. In the mice Nos. 20-1 and 20-2, extremely mild to mild multiple EMH lesions were existed, this did not exclude the possibility of increasing liver EMH when there was liver injury or other hematopoietic injuries. Besides, EMH is also common lesions occurred in spleen of mice and may exist in normal mice, the mild degree of EMH may be therefore considered as background lesions. Extremely mild to mild EMH lesions were found in spleen tissues of all mice. It could be found in the number 25-2 mouse spleen that its lymphoid follicle had multiple extremely mild apoptosis lesions, but no tissue necrosis was found. No obvious degeneration, necrotic or inflammatory cell infiltration lesions were observed in lungs and digestive tissues including stomach, small intestine, colon and pancreas. Chronic progressive nephropathy (CPN) involving lesions of tubule basophilia, basement membrane thickening, mononuclear inflammatory cells infiltration, glomerulosclerosis, or tubule dilatation is the most common kidney lesions in the kidney tissues of mice. The cause of CPN is unknown, it can be found in normal mice, and the incidence is increased depend on ages, male mice have a higher incidence than female mice. Therefore, it is generally not recommended to score CPN lesion separately. CPN may also occur after drug administration, it should be considered as treatment-related lesions if the severities have a significant difference in group differences. In this study, there were moderate or higher CPN lesions in each mouse kidney tissue, except for mouse of No. 25-2. However, the difference was not related to the dose and might not be related to the experimental disposal.

### Example 4: Carbapenem-Resistant Klebsiella pneumoniae Sepsis Infection Model

For establishing the carbapenem-resistant Klebsiella pneumoniae sepsis infection model, 12 mice were separated in each group and were infected with the standard Klebsiella pneumoniae strain ATCC BAA-1705 with lethal dosage by intraperitoneal injection. The compound (c) was given to test groups at the dosage of 0.33, 1.67 and 3.33 mg/kg/day for 3 days treatment, respectively. The combination of emerging combined medications Ceftazidime-Avibactam (200/50 mg/kg/day) was selected as compared drug. 1×PBS was given as placebo group. Mice survival rate were observed four times a day and were recorded as the curve.

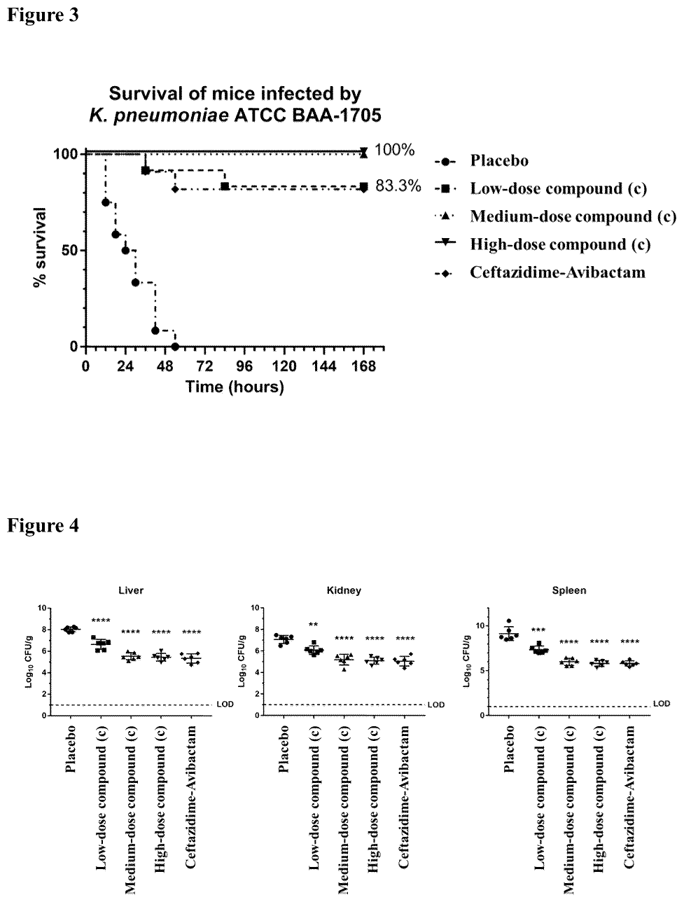

As shown in FIG. 3, mice in the placebo group died within two days. The survival rate of mice receiving Ceftazidime-Avibactam was 83.3% (10/12). The survival rate of mice receiving low-dose (0.33 mg/kg/day) of compound (c) was 83.3% (10/12). The survival rate of mice receiving medium-dose and high-dose (1.67 mg/kg/day and 3.33 mg/kg/day) of compound (c) were 100% (12/12). The results showed that compound (c) had good anti-infection efficacy to septic mice infected with carbapenem-resistant Klebsiella pneumoniae, and the same treating effect were found in mice receiving low-dose of compound (c) and mice receiving Ceftazidime-Avibactam.

The bacteria content in tissues of liver, kidney, and spleen were further analyzed for quantifying the therapeutic effect. Mice were euthanized after one day treatment post-infection, liver, kidney, and spleen were taken and ground, the ground tissues were then serially diluted and coated to agar plates for colonies counting. The difference between each mouse were normalized according to the weight (gram) of each organ.

### Example 5: Carbapenem-Resistant Acinetobacter baumannii Sepsis Infection Model

For establishing the carbapenem-resistant Acinetobacter baumannii sepsis infection model, 12 mice were separated in each group and were infected with the standard Acinetobacter baumannii strain AB03 with lethal dosage by intraperitoneal injection. The compound (c) was given to test groups at the dosage of 0.33, 1.67 and 3.33 mg/kg/day for 3 days treatment, respectively. The clinical standard therapy colistin methanesulfonate (CMS) (40 mg/kg/day) was selected as compared drug. 1×PBS was given as placebo group. Mice survival rate were observed four times a day and were recorded as the curve.

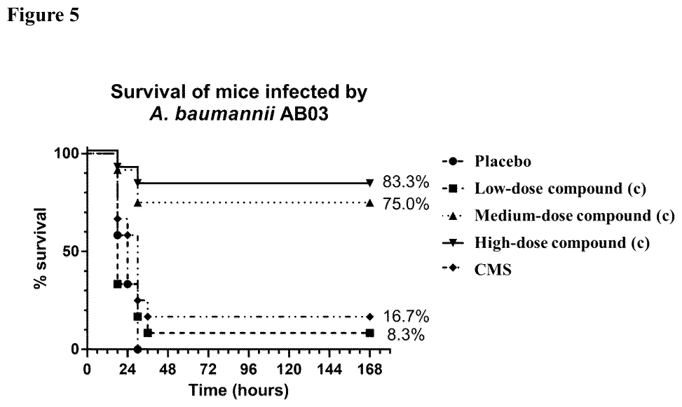

As shown in FIG. 5, mice in the placebo group died within a day. The survival rate of mice receiving CMS was 16.7% (2/12). The survival rate of mice receiving low-dose (0.33 mg/kg/day) of compound (c) was only 8.3% (1/12). However, the survival rate of mice receiving medium-dose and high-dose (1.67 mg/kg/day and 3.33 mg/kg/day) of compound (c) were 75% (8/12) and 83.3% (9/12), respectively. The results showed that medium-dose and high-dose of compound (c) had good anti-infection efficacy to septic mice infected with carbapenem-resistant Acinetobacter baumannii, and the same treating effect were found in mice receiving low-dose of compound (c) and mice receiving CMS.

The present invention has been described and illustrated in sufficient detail to enable those of ordinary skill in the art to which the present invention pertains to understand methods of making and using this art, however, various variations, modifications or improvements are possible and should be deemed to be no different from the spirit and scope of this invention. Those skilled in the art to which the present invention pertains can easily understand and realize the objects of the present invention and obtain the aforementioned results and advantages. The animals and instruments used in the present invention represent the best embodiment, are exemplary, and are not intended to limit the scope of the present invention. Those skilled in the art and the modifications or other uses that will occur when making or using this technology are all included in the spirit of the present invention and defined by the scope of rights.

1. A compound having the following general formula (I): 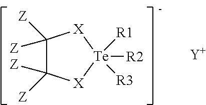
(I),
wherein R1, R2 and R3 are all halogens;
wherein the X is N—R4 or O, and R4 is Hydrogen or alkyl;
wherein the Y is ammonium, phosphonium, potassium, sodium or lithium;
wherein the Z is Hydrogen or Deuterium; and
wherein the Z is not H while the X is O.

2. The compound of claim 1, wherein the compound further forms a pharmaceutical composition by combining with its pharmaceutically acceptable salts.

3. The compound of claim 1, wherein the compound is compound (a) 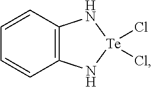
compound (c) 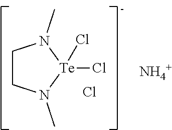
or compound (d)

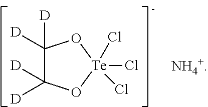

4. The compound of claim 1, wherein the compound is compound (c)

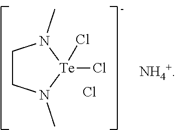

5. (canceled)

6. A use of a pharmaceutical composition in preparation of a method for treating bacterial infections in a subject suffering from bacterial infections, comprising administering an effective amount of compound having the following general formula (I) or a compound having the following general formula (I) and its pharmaceutically acceptable salt thereof: 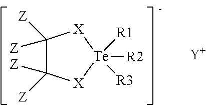
(I),
wherein R1, R2 and R3 are all halogens;
wherein the X is N—R4 or O, and R4 is Hydrogen or alkyl;
wherein the Y is ammonium, phosphonium, potassium, sodium or lithium;
wherein the Z is Hydrogen or Deuterium; and
wherein the Z is not H while the X is O.

7. (canceled)

8. The method of claim 6, wherein the bacterial infections are caused by Gram-negative bacteria.

9. The method of claim 6, wherein the bacterial infections are caused by Klebsiella pneumoniae, Escherichia coli, Pseudomonas aeruginosa, Acinetobacter baumannii, Elizabethkingia meningoseptica, Neisseria gonorrhoeae and/or Enterobacter complex.

10. The method of claim 6, wherein the bacterial infections comprise at least one disease which is selected from one or more of respiratory tract infection, urinary tract infection, central nervous system infection, ear infection, pleuropneumonia and bronchial infection, intra-abdominal infection, cardiovascular infection, skin or soft tissue infections, bone and joint infections, genital infection, eye infection, pharyngeal infection, and oral infection.

11. The method of claim 6, wherein the bacterial infections comprise at least one disease which is selected from one or more of upper respiratory tract infection, lower respiratory tract infection, tracheitis, bronchitis, pneumonia, pulmonary tuberculosis, pharyngitis, complicated urinary tract infection, non-complicated urinary tract infections, cystitis, pyelonephritis, encephalitis, meningitis, brain abscess, otitis externa, otitis media, blood infection, endocarditis, myocarditis, pericarditis, arthritis, osteomyelitis, genital ulceration, vaginitis, cervicitis, conjunctivitis, keratitis, endophthalmitis, and gingivitis.

12. The method of claim 11, wherein the blood infection is sepsis or bacteremia.

13. A compound having the following general formula (II): 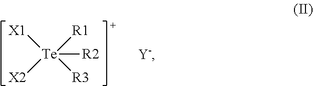
wherein R1, R2 and R3 are all halogens;
wherein Y is halogen; and
wherein the X1 and X2 are together bipyridine, dipyridinyl disulfide or phenanthroline, wherein the bipyridine is unsubstituted or substituted by one or more alkyl substituents.

14. The compound of claim 13, wherein the compound further forms a pharmaceutical composition by combining with its pharmaceutically acceptable salts.

15. The compound of claim 13, wherein the compound is compound (b) 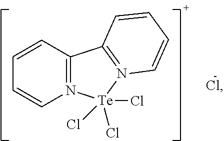
compound (e) 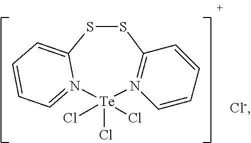
compound (f) 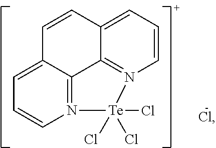
compound (g) 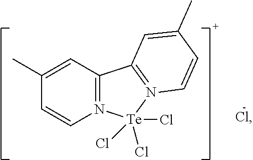
or compound (h)

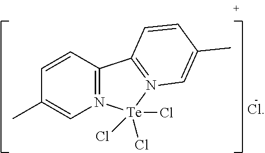

16. (canceled)

17. A method for treating bacterial infections in a subject suffering from bacterial infections, comprising administering an effective amount of compound having the following general formula (II) or a compound having the following general formula (II) and its pharmaceutically acceptable salt thereof: 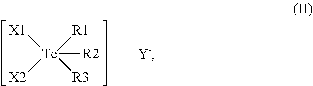
wherein R1, R2 and R3 are all halogens;
wherein Y is halogen; and
wherein the X1 and X2 are together bipyridine, dipyridinyl disulfide or phenanthroline, wherein the bipyridine is unsubstituted or substituted by one or more alkyl substituents.

18. (canceled)

19. The method of claim 17, wherein the bacterial infections are caused by Gram-negative bacteria.

20. The method of claim 17, wherein the bacterial infections are caused by Klebsiella pneumoniae, Escherichia coli, Pseudomonas aeruginosa, Acinetobacter baumannii, Elizabethkingia meningoseptica, Neisseria gonorrhoeae and/or Enterobacter complex.

21. (canceled)

22. The method of claim 17, wherein the bacterial infections comprise at least one disease which is selected from one or more of respiratory tract infection, urinary tract infection, central nervous system infection, ear infection, pleuropneumonia and bronchial infection, intra-abdominal infection, cardiovascular infection, skin or soft tissue infections, bone and joint infections, genital infection, eye infection, pharyngeal infection, and oral infection.

23. The method of claim 17, wherein the bacterial infections comprise at least one disease which is selected from one or more of upper respiratory tract infection, lower respiratory tract infection, tracheitis, bronchitis, pneumonia, pulmonary tuberculosis, pharyngitis, complicated urinary tract infection, non-complicated urinary tract infections, cystitis, pyelonephritis, encephalitis, meningitis, brain abscess, otitis externa, otitis media, blood infection, endocarditis, myocarditis, pericarditis, arthritis, osteomyelitis, genital ulceration, vaginitis, cervicitis, conjunctivitis, keratitis, endophthalmitis, and gingivitis.

24. The method of claim 23, wherein the blood infection is sepsis or bacteremia.
